(19) United States
(12) Patent Application Publication
(10) Pub. No.: US 2017/0197051 A1
(43) Pub. Date: Jul. 13, 2017

(54) Novel Airway Tube Stabilizer

(71) Applicant: David Kumpel, Dyer, IN (US)

(72) Inventor: David Kumpel, Dyer, IN (US)

(21) Appl. No.: 15/472,732

(22) Filed: Mar. 29, 2017

Related U.S. Application Data
(63) Continuation-in-part of application No. 14/882,184, filed on Oct. 13, 2015.
Application No. 14/882,184 is a continuation-in-part of application No. 62/063,514, filed on Oct. 14, 2014.

(51) Int. Cl. A61M 16/04

(52) U.S. Cl. CPC A61M 16/0497; A61M 16/0493; A61M 2202/0208; A61M 2205/0216

(57) ABSTRACT

A novel tube stabilizer with a collar and an aperture. A tube from a medical airway device can pass through the aperture. The laryngeal mask stabilizer slides along the tube and fits into the mouth of a patient on which an airway device is being used during a medical procedure. The apparatus stabilizes the airway tube.

### CROSS-REFERENCE TO RELATED APPLICATIONS

The present application is a continuation-in-part of application Ser. No. 14/882,184, filed Oct. 10, 2015 which is a non-provisional application that claimed the benefit of U.S. Provisional Patent Application No. 62/063,514, filed Oct. 14, 2014, the entireties of which are hereby incorporated by reference.

### BACKGROUND

An airway device, such as a supra-glottic airway, is used by medical professionals and anesthesia providers for establishing breathing paths, airways, in unconscious patients. These are well know to anesthesia providers and emergency personnel worldwide. They are also used for administering and monitoring anesthesia in unconscious patients. Airway devices can also be used in emergency situations when ventilation is necessary. Generally, an airway device comprises a sealing portion and an airway tube which work together to establish an airway in a patient, intubation. They are used for one goal, to establish an open, or patent, airway in a person or animal.

In the operating room or surgical setting, the supraglottic airway is inserted into the mouth and placed deep enough to seat or rest in the larynx, above the glottis. The end of the supraglottic airway is then attached to an anesthesia breathing circuit, to deliver oxygen, air, or inhalational agent such as anesthesia gas—or all three.

In an emergency setting, a trained provider, such as a Nurse Anesthetist, Anesthesiologist, Advanced Practice Nurse, or Emergency Medical Technician will use the supraglottic airway to establish a patent or open airway in a patient who is not breathing or having difficulty breathing on their own. In this case, oxygen or room air is delivered through the supraglottic airway to help save the patient's life.

These airway devices have worked well over the years but problems still occur during their use. To keep a reliably working airway in a patient, it is crucial to have a good seal around the laryngeal inlet or the glottis at all times during use of the device. This means that the airway device must stay in position, remaining stable, throughout the patient's surgery or other medical procedure. This stability becomes difficult to maintain when the patient involuntarily moves while anesthetized. Such movements cause the airway device to become dislodged or to shift positions which causes a breaking of the path for air.

Use of an intubation tube on patients having few or no teeth poses additional stability problems. An edentulous person has extra space in their mouth from the lack of teeth that used to occupy this space. When the supraglottic airway is inserted into the airway of an edentulous person, it is loose or ill-fitting because of the lack of teeth to support or surround the supraglottic airway.

Loss of a patient's airway is a life threatening situation. It is well known that a poorly fitted supra-glottic airway is not only unsafe, but can become a medical emergency if oxygen cannot be delivered safely to a patient. In fact, the number one cause of anesthesia related deaths is airway emergencies becoming airway disasters resulting in patient deaths.

Additionally, the loss of stability in an airway device causes distractions during the medical procedure because of the need for airway device adjustments to get the patient breathing again. Such distractions are time-consuming, difficult for medical staff and cause great danger to the patient. There is therefore a need for a device that can keep an airway device stably in place during medical procedures which involve the use of an airway device for keeping a patient's airway clear.

Information relevant to attempts to address these problems can be found in U.S. Pat. No. 7,134,431 B2. However, this reference suffers from one or more of the following disadvantages: the tab method does not provide enough surface area to benefit from the patient's body support and the tab does not work well on edentulous patients. Also, tape around the mouth and chin is also used to provide stabilization of the tube but tape can lose its adhesiveness and the tube still fits poorly and requires adjusting, which can cause dislodging.

There is a need in the industry for an apparatus that is easily used with an airway device, takes advantage of the patient's body for support, and stabilizes the airway device. The need is accentuated with respect to patients that do not have teeth and there is an additional necessity to fill the void left by missing teeth that might otherwise assist in stabilizing an airway device.

### SUMMARY

The present invention is directed to an apparatus that satisfies this need. I have invented a novel airway tube stabilizer comprising a collar and an aperture. A preferred embodiment has its collar being made from a soft medical grade thermoplastic elastomer (TPE). The collar fits and finds support from a patient's mouth area. In one embodiment, it fits snugly around a laryngeal mask and is placed just under the upper and lower lip of a patient. A tube from an airway device, such as that used on human patients in medical procedures or on animals in veterinary procedures, for example a laryngeal tube, or the like, easily passes into and through the aperture allowing for easy positioning of the apparatus. The apparatus is especially useful for providing stability to airway devices when used on edentulous patients however it can be used anytime an airway device with a tube inserted into the patient's throat is used. The stability provided by the apparatus can be achieved through its shape, to name one example of how the apparatus provides stability to a tube.

Another embodiment of the present invention can be used as a stabilizer for an airway tube used on a medical patient. The tube is preferred to be a laryngeal tube but certainly can work on any other tube that is inserted into the mouth of a patient that seals within against the patient's body to provide an unobstructed path, for example a laryngeal tube providing a clear path for oxygen to pass into a patient's trachea. The embodiment includes a collar segment, it somewhat resembles a mouthpiece that is used in sporting activities. The collar segment has at least one first opening that is generally in the center of the collar segment because that is where the collar segment material provides the most lateral support to the walls of the first opening, or aperture. The at least one first opening can receive the airway tube, the airway tube can pass through the opening. When used the collar segment is removably attached to an airway tube. Preferably this is done by passing the tube through the first opening and the collar segment sliding along the tube. However, the collar segment could also have a snap or hinged design that could allow the collar segment to be placed onto the tube without having to remove the oxygen circuit. The collar segment engages the patient's lips by being placed generally under the upper and lower lips. This is especially useful when the patient has no teeth. Because patients will vomit or expel other material while in surgery or need to have other medical apparatus' inserted into the oral cavity, the collar segment may have at least one second opening or be configured such that there is a means to use the mouth opening to aspirate. The path of for oxygen that the tube takes generally traverses the patient's oral cavity, seats in or goes through the patient's larynx, and then could continue up to the patient's trachea. Oxygen can then make its way into the patient's lungs.

The collar segment prevents the airway tube from moving and breaking the clear path for oxygen to pass. This is because there is stability in the design of the collar segment that secures the tube and prevents it from becoming dislodged or needing readjustments. The stabilizer is best made for only a single use and manufactured from a TPE material.

Another embodiment of the present invention works as an intubation tube positioning device that is used on medical patients, human or animal. The device includes a collar segment that has a medial aperture. That medial aperture is configured to accept an intubation tube. The collar segment is shaped to accommodate upper and lower lips of a patient, and finds some support from the patients gums.

REDO - - - A novel method embodiment of my invention includes a positioning and stabilizing a laryngeal intubation tube that is part of a supra-glottic mask, often times referred to as a laryngeal mask. The tube is positioned centrally in a patient's mouth during intubation, where intubation is not being limited to endotracheal tube utilization but also includes application of a laryngeal mask, through the use of an intubation mouthpiece. The mouthpiece includes a medial aperture configured to accept an end of the intubation tube. This method for using it involves advising the patient to go to sleep before they undergo the procedure. Then covering the patient's mouth with an oxygen mask connected to a breathing circuit, machinery common in the anesthesia industry, that delivers oxygen. Then delivering anesthesia to the patient via an IV to put the patient to sleep. After the patient falls asleep and is no longer breathing, when apnea sets in, then the user removes the circuit and oxygen mask that is covering the patient's mouth. Using fingers to scissor the patient's mouth open, the laryngeal intubation tube that is part of a supra-glottic mask is inserted into the patient via the patient's mouth to create a seal between the supra-glottic mask and the patient's body just above the patient's glottis. Then the user attaches the circuit to the laryngeal intubation tube that is part of a supra-glottic mask and checks that the patient's chest is rising to indicate that oxygen is entering the patient's lungs, indicating a positive end tidal CO2. Next is to quickly disconnect the circuit from the laryngeal intubation tube that is part of a supra-glottic mask. Then attaching the mouthpiece to fit snugly around the laryngeal intubation tube that is part of a supra-glottic mask by passing the exposed end of the tube through the medial aperture. Then the use will reconnect the circuit to the laryngeal intubation tube that is part of a supra-glottic mask. Then sliding the mouthpiece so that is sits under the patient's upper and lower lips providing sufficient support to the laryngeal intubation tube that is part of a supra-glottic mask to prevent breaking the seal. If necessary, the user can continue by taping the patient's chin and mouth to provide added support to the laryngeal intubation tube that is part of a supra-glottic mask.

REDO

Another embodiment of the present invention is an intubation system used on medical patients. It comprises a laryngeal intubation tube being inserted into a larynx with a tube end protruding from the mouth of a patient. There is an intubation tube mouthpiece configured to fit between upper and lower lips of the patient. The intubation tube mouthpiece includes a medial aperture sized to fit the intubation tube, wherein the tube end passes through the medial aperture and the mouthpiece is seated between the upper and lower lips of the patient. The intubation tube mouthpiece having at least one second aperture providing access to and from the patient's oral cavity. This system can have the mouthpiece made for only a single use made from TPE material or the like.

The material should be a soft pliable material that is comfortable to use yet is sturdy. The material should maintain its shape when manipulated and is made of such a material as to sufficiently allow the invention to meet the requirements of providing stability to airway devices used on patients in surgery as discussed herein.

One embodiment of the present invention relates to a novel tube stabilizer used, for example, as an accessory to airway devices and more particularly to a medical device having a collar disposed around an airway device's tube, or the like, for controlling the airway device's position and more specifically to such a stabilizer in which the collar with a hole that is adapted to be slidable along the tube of the airway device and sufficiently fitting the rima oris of a patient being ventilated.

Another embodiment of the airway tube stabilizer, which I refer to as a laryngeal mask stabilizer, comprises a holder that is generally circular in shape. The holder has a hole through its midsection, a medial aperture, with at least one grip. The at least one grip is integrally attached to the holder. It is envisioned that the invention is injection molded. The at least one grip attached along a surface that bounds the medial aperture.

There is a support that has a proximal and a distal end. The support has at least one restrictor and a seat. The support being integrally attached to the holder at its proximal end with the distal end being situated opposite the holder. This creates a sort of platform that extends out from the holder.

The at least one restrictor is integrally attached to a lateral portion of the support near the proximal end. The seat too is integrally attached to a top surface of the support.

The medial aperture capable of receiving an airway tube. In practice, the airway tube would pass through the medial aperture, under a bottom side of the support and into the mouth of a patient.

The seat fits snugly in an upper palate of a mouth. A portion of the holder can fit between upper and lower gums of a mouth on a patient. In practice, a portion of the holder will between the gums and a portion will be on either side of the gums, inside or outside of the mouth. The restrictor is appendage like and presses against the inside surface of a mouth to prevent rotation of the airway tube.

The size of one embodiment of the invention can be made to vary in several respects depending on the size of the person or animal being treated. Additionally, the invention can be made with second holes or without. In the latter situation, the access to a patient's mouth is done by using the openings between the invention and the side of the patient's mouth.

### BRIEF DESCRIPTION OF THE SEVERAL VIEWS OF THE DRAWINGS

These and other features, aspects, and advantages of the present invention will become better understood with regard to the following description, appended claims, and accompanying drawings where:

FIG. 1 shows a perspective view of one embodiment of the invention.

FIG. 2 shows a perspective view of another embodiment of the invention being used with an intubation tube.

FIG. 3 shows a perspective view of another embodiment of the invention.

FIG. 4 shows a head on view of another embodiment of the invention.

FIG. 5 shows a view of another embodiment of the invention being used with an intubation tube.

### DESCRIPTION

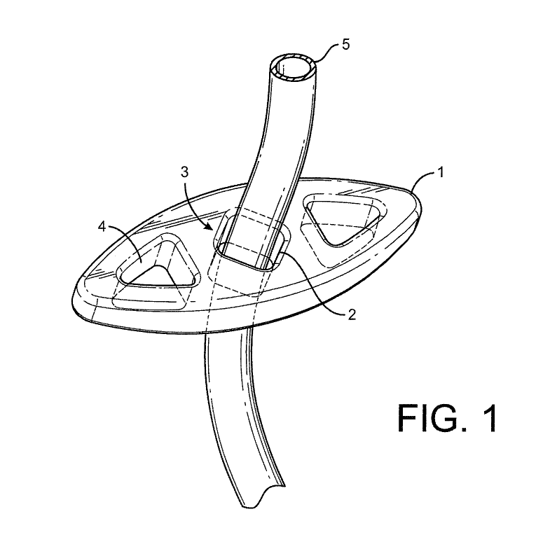

As shown in FIG. 1, a perspective view of one embodiment of the invention comprises a collar 1, the collar 1 having an aperture 2. An airway tube 5 passing through the aperture 2. The aperture 2 keeping a snug fit on the airway tube 5 but yet allowing for sliding. The collar 1 having at least one hole 4. The aperture 2 is shaped 3 to accommodate differently sized airway tubes 5.

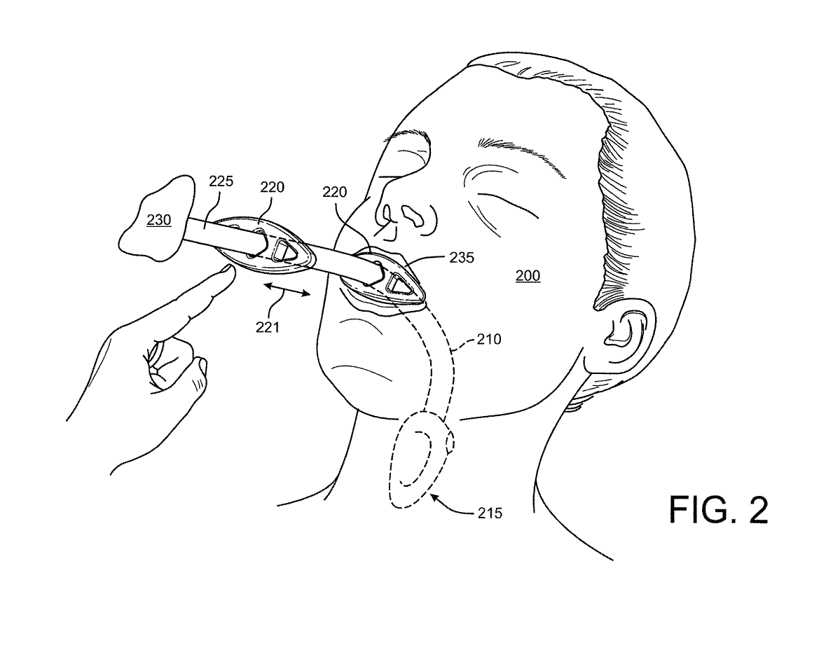

As shown in FIG. 2, a perspective view of another embodiment of the invention being used with an airway device comprises a patient 200 that has been fitted with an airway device 210. The airway device 210 seals around the patient's laryngeal opening or glottis 215. The airway device having a tube 225 that travels outside of the patient to ventilation equipment 230. The collar 220 being moveable 221 along the tube 225. Generally a medical provider will slide the collar 221 into position. In one position the collar 220 is fit into and around the patient's mouth 235 under the patient's 200 lips filling the void and stabilizing the airway device 210.

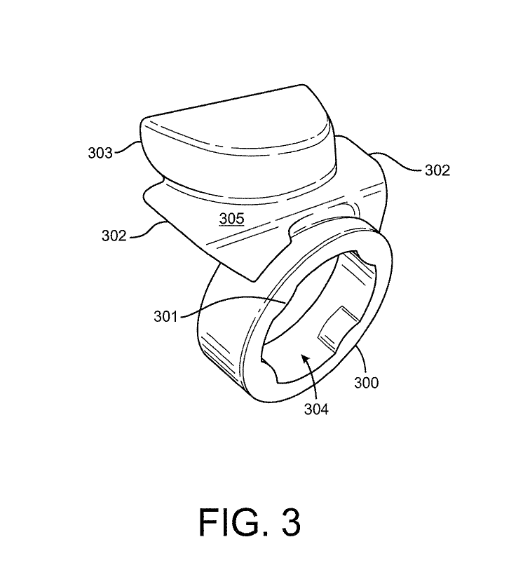

A shown in FIG. 3, a perspective view of another embodiment of the invention comprises a holder 300 that is integrally attached to a support 305. The holder 300 having at least one grip 301 located around a medial aperture 304 of the holder 300. There is a seat 303 portion of the support 305. This seat portion would fit an upper palate of a patient (not shown). The support having at least one restrictor 302 which restricts the movement of the invention when in use. The at least one restrictor located at the end of the support proximal to the holder 300.

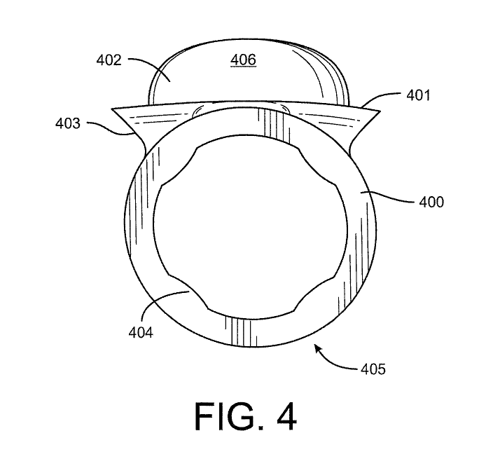

As shown in FIG. 4 a head on view of another embodiment of the invention 405 which comprises a holder 400. The holder being integrally attached to a support 406 having a seat 402 and a plurality of restrictors 401 and 403. The holder 400 has at least one grip 404 located around a medial aperture.

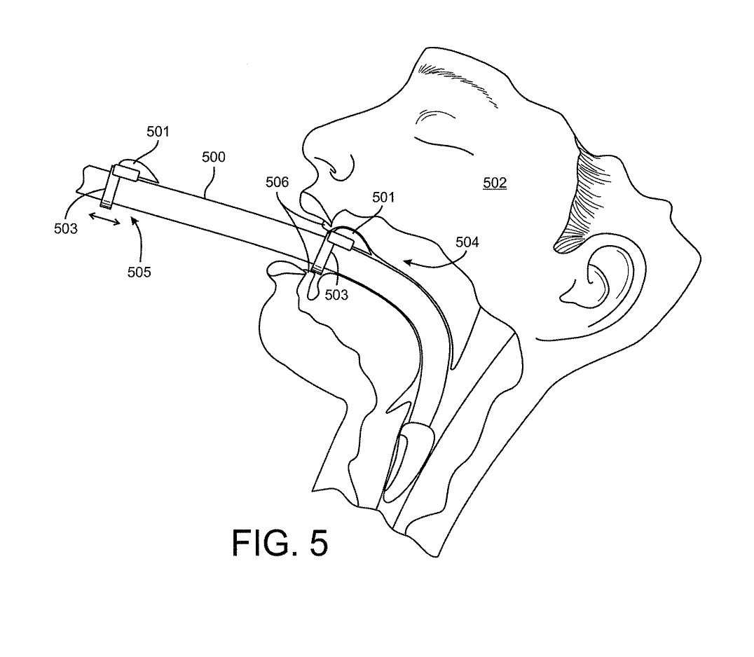

As shown in FIG. 5 another embodiment of the invention being used with an intubation tube comprises the invention slidably secured 505 to an intubation tube 500 which is inserted into a human patient 502. The tube 500 fits through a medial aperture of the holder 503. A support 501 is integrally attached to the holder. When the holder 503 is slid into the patient's 502 mouth 504 the support 501, being shaped like a wedge, fits into the palate of the patient's 502 mouth. The holder 503 is located between the patient's gums 506.

Although the present invention has been described in considerable detail with the reference to certain preferred versions thereof, other versions are possible. For example, the collar of the invention could be hinged or semi-closed and allow for opening to fit around a tube or being pressed to snap onto the tube instead of being slid over the tube. Therefore, the spirit and scope of the appended claims should not be limited to the description of the preferred versions contained herein.

Any element in a claim that does not explicitly state “means for” performing a specified function, or “step for” performing a specific function, is not to be interpreted as a “means” or “step” clause as specified in 35 U.S.C. §112, ¶6. In particular, the use of “step of” in the claims herein is not intended to invoke the provisions of 35 U.S.C. §112, ¶6.

What I claim is:

1. A stabilizer for an airway tube used on a medical patient, the stabilizer comprising:
a collar segment;
the collar segment being made of a pliable material;
the collar segment having at least one first opening;
the at least one first opening capable of receiving the airway tube;
the collar segment being removably attached to the airway tube;
the collar segment engaging the patient's lips;
the collar segment having at least one second opening;
the airway tube being one that is commonly used in medical procedures when the patient requires a clear path for oxygen to pass, the path generally traversing the patient's oral cavity, then through the patient's larynx, and then into the patient's trachea;
the collar segment preventing movement of the airway tube which would cause breaking of the clear path for oxygen to pass.

2. The stabilizer of claim 1 wherein the stabilizer is made for only a single use.

3. The stabilizer of claim 1 wherein the pliable material is TPE.

4. An intubation tube positioning device used on medical patients, the device comprising:
a collar segment;
the collar segment having a medial aperture;
the medial aperture configured to accept an intubation tube;
the collar segment being shaped to accommodate a space under the upper and lower lips of a patient.

5. A method of positioning and stabilizing a laryngeal intubation tube that is part of a supra-glottic mask centrally in a medical patient's mouth during intubation utilizing an intubation mouthpiece, the mouthpiece including a medial aperture configured to accept an end of the laryngeal tube, the method comprising:
preparing a medical patient to be put to sleep before a medical procedure that requires the use of anesthesia;
covering the patient's mouth with an oxygen mask connected to a breathing circuit that delivers oxygen;
delivering anesthesia to the patient via an IV to put the patient to sleep;
after the patient falls asleep and is no longer breathing then removing the breathing circuit and the oxygen mask that is covering the patient's mouth;
using fingers to open the patient's mouth, the laryngeal intubation tube that is part of a supra-glottic mask is inserted into the patient via the patient's mouth to create a seal between the supra-glottic mask and the patient's body just above the patient's glottis;
attaching the oxygen circuit to the laryngeal intubation tube that is part of a supra-glottic mask and checking that the patient's chest is rising to indicate that oxygen is entering the patient's lungs, indicating a positive end tidal CO2;
quickly disconnecting the oxygen circuit from the laryngeal intubation tube that is part of a supra-glottic mask;
attaching the intubation mouthpiece to fit snugly around the laryngeal intubation tube that is part of a supra-glottic mask by passing the exposed end of the intubation tube through the medial aperture;
reconnecting the circuit to the laryngeal intubation tube that is part of a supra-glottic mask;
sliding the mouthpiece so that it sits under the patient's upper and lower lips providing sufficient support to the laryngeal intubation tube that is part of a supra-glottic mask to prevent breaking the seal;
taping the patient's chin and mouth to provide additional support to the laryngeal intubation tube that is part of a supra-glottic mask.

6. An intubation system used on medical patients, the system comprising:
a laryngeal intubation tube;
the intubation tube being inserted into a larynx with a tube end protruding from the mouth;
an intubation tube mouthpiece configured to fit between upper and lower lips of the patient;
the intubation tube mouthpiece including a medial aperture sized to fit the intubation tube, wherein the tube end passes through the medial aperture and the mouthpiece is seated under the upper and lower lips of the patient;
the intubation tube mouthpiece having at least one second aperture;
the at least one second aperture providing access in and out of the patient's oral cavity.

7. The stabilizer of claim 6 wherein the mouthpiece is made for only a single use.

8. The stabilizer of claim 6 wherein the mouthpiece is made from TPE material.

9. A novel airway tube stabilizer comprising:
a holder;
the holder having a medial aperture;
at least one grip;
the at least one grip being integrally attached to the holder along a surface bounding the medial aperture;
a support;
the support having a proximal end;
the support having a distal end;
the support having at least one restrictor;
the support having a seat;
the proximal end being integrally attached to the holder;
the distal end being situated opposite the holder;
the at least one restrictor being integrally attached to a lateral portion of the support near the proximal end;
the seat being integrally attached to a top surface of the support;
the medial aperture capable of receiving an airway tube.

10. The apparatus of claim 9 wherein the seat fits snugly in an upper palate of a mouth.

11. The apparatus of claim 9 wherein a portion of the holder fits between upper and lower gums of a mouth.

12. The apparatus of claim 9 wherein the at least one restrictor presses against the inside surface of a mouth to prevent rotation of the airway tube.

13. A laryngeal mask stabilizer comprising:
a support structure disposed in a patient's mouth, the support structure including:
a convex seat element configured to engage an upper palate curvature;
one or more restrictors extending outward from a periphery of the seat element, the one or more restrictors bearing on an inner cheek wall;
the support structure contacting the upper palate and the inner cheek so that the airway tube maintains a central location with respect to the mouth; and
a tube holder connected to the support structure including a medial aperture configured to accept an airway tube extending upwards from the patient's larynx and exiting the mouth through the medial aperture.

14. The laryngeal mask stabilizer of claim 13, wherein the one or more restrictors are two restrictors extending symmetrically opposite each other around the periphery and wherein the two restrictors bear evenly on opposite cheek walls.

15. A method of stabilizing an airway tube of an intubated patient with a laryngeal mask stabilizer including a convex seat element, restrictors extending outward from a periphery of the seat element, and a tube holder having a medial aperture, the method comprising:
placing the laryngeal mask stabilizer into the patient's mouth;
positioning the convex seat element against an upper palate so that the restrictors bear on opposite inner cheek walls and the medial aperture is disposed between lips of the patient; and
insert an airway tube through the medial aperture so that the tube extends from a larynx and exits the mouth.
